(19) United States
(12) Patent Application Publication
(10) Pub. No.: US 2019/0162731 A1
(43) Pub. Date: May 30, 2019

(54) METHOD FOR MONITORING FETUS/PRETERM INFANT DEVELOPMENT, AND FOR PROMOTING THE NORMAL DEVELOPMENT OF PRETERM INFANTS

(71) Applicant: Zietchick Research Institute, LLC, Plymouth, MI (US)

(72) Inventor: Tammy Z. Movsas, Southfield, MI (US)

(73) Assignee: Zietchick Research Institute, LLC, Plymouth, MI (US)

(21) Appl. No.: 16/096,258

(22) PCT Filed: Apr. 27, 2017
(86) PCT No.: PCT/US17/29880

§ 371 Date: Oct. 24, 2018

Related U.S. Application Data
(60) Provisional application No. 62/328,345, filed on Apr. 27, 2016.

(51) Int. Cl. G01N 33/68

(52) U.S. Cl. CPC G01N 33/68; G01N 2800/7014; G01N 2800/38; G01N 2333/515

(57) ABSTRACT

A method for monitoring fetal and/or preterm infant development and a method to promote normal growth & development of preterm infants, especially in regards to each preterm infant's collective set of immature organs. Monitoring development is accomplished by using VEGF 121 as a biomarker in bodily fluid levels, to determine whether appropriate angiogenic activity is occurring to allow preterm infant or fetal development to proceed normally. The promotion of preterm infant normal development is accomplished by administering to a preterm infant human chorionic gonadotropin (hCG) and/or Luteinizing hormone (LH) and/or Luteinizing hormone releasing hormone (LHRH) in physiological amounts at appropriate intervals to raise and maintain the activation level of the patient's combined hCG/LH receptor activity, or VEGF 121 level, to a level that is normally present in fetuses of the equivalent developmental (gestational) age. By promoting normal development, we prevent onset or progression of disorders associated with premature organs rather than treat disorders associated with the premature organs after they occur.

### CLAIM OF PRIORITY

This application claims priority to provisional patent application Ser. No. 62/328,345, filed Apr. 27, 2016, and entitled HORMONAL TREATMENT FOR RETINOPATHY OF PREMATURITY.

### FIELD OF THE INVENTION

The technology described herein relates generally to the field of treating premature infants.

### BACKGROUND OF THE INVENTION

The preterm birth (also referred to as premature birth) of a human infant is defined as birth before a gestational age of 37 complete weeks. When infants are born preterm, they are born before many of their organ systems have reached full maturity. Preterm infants show physical signs relating to their premature organs in reverse proportion to their gestational age. In other words, the more preterm an infant is born, the more premature the organs are at birth and the higher the likelihood that the organs will not mature normally outside the womb and thus, begin to develop disorders associated with prematurity.

The organs of the body rely upon the 38-40 weeks of normal intrauterine life for complete maturation; thus, any organ system can be affected by preterm birth (visual, neurologic, gastrointestinal, cardiovascular, pulmonary, hematologic, immune, etc.). Thus, preterm infants are at risk for a large variety of morbidities which can affect any organ of the body. The morbidities of prematurity are especially severe for infants born before the 34^{th} week of gestation and/or under 2000 g.

Some of these disorders are specific to premature infants such as retinopathy of prematurity (ROP) and bronchopulmonary dysplasia. Other disorders are not specific to prematurity but are more common in children who had been born preterm. A few of the many examples of these disorders are asthma, attention deficit disorder, cerebral palsy, autism and patent ductus arteriosus. Other disorders, related but not restricted to prematurity, do not present themselves until adulthood. For example, the odds of acquiring high blood pressure and diabetes mellitus are significantly greater in adult survivors of extreme prematurity than in adults who had been born of normal gestational age. In addition, recent studies show an increase in cardiac abnormalities in adult survivors of extreme prematurity; most of these cardiac abnormalities were not detected during childhood. (Reference: Doyle L W, Anderson P J. Adult outcome of extremely preterm infants. Pediatrics. 2010;126(2):342-351)

Whereas full term infants are born with mature retinas, preterm infants are born with retinas that are not yet fully vascularized. Approximately 10% of annual births worldwide (13 million infants/year) are preterm, placing these infants at risk for the potentially blinding disorder called retinopathy of prematurity (ROP) or formerly known as retrolental fibroplasia. Though all preterm infants are at risk for ROP, those whose birthweights are <1500 g and/or whose gestational ages are <30 weeks, are at highest risk.

Early stage ROP (Stage 1, Stage 2) occurs when the vascularization process of the preemie's retina is prematurely arrested. At this point, ROP can regress if normal vascularization re-initiates itself, or the retina can become ischemic. {Scott A, Fruttiger M. Oxygen-induced retinopathy: a model for vascular pathology in the retina. Eye (Loud). March 2010;24(3):416-421.} If and when ROP advances to Stages 3-5, then retinal vascularization proceeds pathologically via a process known as neovascularization (the growth of fragile, abnormal retinal vessels). Neovascularization may be accompanied by the formation of fibrous tissue which can lead to scarring and retinal detachments. Most treatments for ROP are currently administered to infants with late stage disease. For example, late stage ROP is generally treated by peripheral retinal ablation via laser photocoagulation or cryotherapy. Anti-VEGF agents are currently being investigated in preterm infants with late stage of retinopathy of prematurity (ROP). In our WO 2016/010786 A1 we treat (late stage) ROP with hCG antagonists and/or LH antagonists.

Less attention has been devoted to identifying agents that enhance normal vascularization of the preterm infant's retina to prevent ROP from occurring or preventing ROP from advancing from early stage to late stage. One such potential agent for mitigating ROP is BIBF1120 (Vargatef) which has been shown to inhibit preretinal neovascularization and enhance normal vascularization in a rat model of vaso-proliferative retinopathy. [Rivera J C, Noueihed B, Omri S, Barrueco J, Hilberg F, Chemtob S. BIBF1120 (Vargatef) Inhibits Preretinal Neovascularization and Enhances Normal Vascularization in a Model of Vasoproliferative Retinopathy. Invest Ophthalmol Vis Sci. 2015;56(13):7897-7907.]

US Published Patent Application 2008/0317721, to Friedlander et al, published on Dec. 25, 2008, discloses a method for treating retinopathy of prematurity and related retinopathic disease. The method comprises administering to the retina of a mammal suffering from, or at risk of developing ROP or a related retinopathy disease an amount of cells from a vasculotrophic lineage negative hematopoietic stem cell population, to promote beneficial physiological revascularization of damaged areas of the retina and to ameliorate damage to the retina caused by the disease.

Thus far, the only effective treatment for promoting overall normal development are treatments that help prevent preterm birth itself. There are some treatment options for some of the morbidities of prematurity. For example, surfactant is now standard of care to promote lung maturation. That said, bronchopulmonary dysplasia is still a common occurrence. When retinopathy of prematurity (ROP) was first reported in the 1940's, it was highly associated with unregulated oxygen delivery. Drastic reductions in oxygen delivery to preterm infants has certainly helped lower ROP rates. However, ROP continues to be a common occurrence in preterm infants, especially in low birthweight infants.

In our prior publication, Use Of Human Chorionic Gonadotropin To Treat Cerebral Palsy And/Or Its Co-Morbidities, WO 2014116786 A1, the use of human chorionic gonadotropin (hCG) was suggested to prevent cerebral palsy and other disorders that result from brain insults (such as hypoxia-ischemia) leading to brain damage. The co-morbidities that are included in this prior patent refer to the co-morbidities of cerebral palsy which are all due to brain damage. This prior art does NOT refer to the co-morbidities of prematurity. For example, cerebral palsy is associated with strabismus (crossed eyes) These is a types of eye problem that arise from the same type of brain damage from can cause cerebral palsy and NOT from the development of the eye itself. Retinopathy of prematurity, which as mentioned above as a disorder of prematurity, is due to the lack normal development of the retinal vasculature and not due to brain damage. Thus, retinopathy of prematurity (ROP), unlike strabismus, is not co-morbidity of cerebral palsy (CP). Co-morbidities of prematurity are NOT co-morbidities of CP. For example, ROP, autism spectrum disorder, patent ductus arteriosus, bronchopulmonary dysplasia and CP are all more common in infants who were born prematurely. However, neither ROP nor autism spectrum disorder nor bronchopulmonary dysplasia, nor patent ductus arteriosus are considered to be co-morbidities of cerebral palsy. On the other hand, ROP, autism spectrum disorder, patent ductus arteriosus, bronchopulmonary dysplasia and CP are all considered to be co-morbidities of prematurity.

There is evidence that Vascular Endothelial Growth Factor (VEGF), also referred to as VEGF A, is involved in physiologic vasculogenesis and angiogenesis and is essential for normal human growth and development. In mouse models, inactivation of a single VEGF allele results in embryonic lethality between day 11 and day 12. (Vascular Endothelial Growth Factor: Basic Science and Clinical Progress, Napoleone Ferrara, Endocrine Reviews 25(4): 581-611). The human VEGF (or VEGF A) gene consists of 8 exons. Alternative splicing can generate several VEGF isoforms. Four main isoforms have been described in humans: VEGF 121, VEGF165, VEGF 189 and VEGF206. Besides the isoforms of VEGF that promote angiogenesis and vasculogeneis, there are some isoforms of VEGF that promote the opposite: anti-angiogensis. Of note, human chorionic gonadotropin and luteinizing hormone have been described as angiogenic VEGF regulators in several reproductive organs (such as the gravid uterus) as well as in certain tumor types.

VEGF A (which refers either to a combination of VEGF A isoforms or refers to the VEGF165 isoform by itself) has been evaluated as a potential biomarker for preterm infant health as well as pregnancy status. Low urinary VEGF levels (when isoforms 121 and 165 are measured collectively or when VEGF 165 is measured alone) are associated with some of the co-morbidities of prematurity (such as bronchopulmonary dysplasia or ROP) [REF: Low urine vascular endothelial growth factor levels are associated with mechanical ventilation, bronchopulmonary dysplasia and retinopathy of prematurity, Bernadette M Levesque, Leslie A Kalish, et al. Neonatology 2013; 104;56-64] However, there has never been a consistency in VEGF levels in preterm infants to allow for the measurement of either VEGF A (referring to a measurement of various combination of different VEGF A isomers as a whole) or VEGF165 (which is known in the literature as the most common VEGF isomer) to serve as a biomarker for fetal growth or development or preterm infant growth and development.

### SUMMARY OF THE INVENTION

Rather than treating specific conditions or individual morbidities of prematurity, the present invention is a method for monitoring fetal and/or preterm infant development and a method to promote normal growth & development of preterm infants, especially in regards to each preterm infant's collective set of immature organs, in order to prevent onset or progression of disorders associated with premature organs, and thereby avoid having to treat such disorders.

Monitoring development is accomplished by using VEGF 121 as a biomarker. Bodily fluid levels of VEGF121 are monitored, to determine whether appropriate angiogenic activity is occurring to allow preterm infant or fetal development to proceed normally. The bodily fluid level of VEGF 121 in a fetus is determined indirectly, by determining bodily fluid levels of VEGF121 in the pregnant mother, which would correspond to fetal level.

The promotion of preterm infant normal development is accomplished by administering to a preterm infant human chorionic gonadotropin (hCG) and/or Luteinizing hormone (LH) and/or Luteinizing hormone releasing hormone (LHRH) in physiological amounts at appropriate intervals to raise and maintain the activation level of the patient's combined hCG/LH receptor activity, or VEGF 121 level, to a level that is normally present in fetuses of the equivalent developmental (gestational) age. The preterm infant should be treated until his/her organs reaches the equivalent maturity of the organs of full term infants. In this patent application, hCG, LH and LHRH will individually or collectively be referred to as the LH/hCG receptor activator.

The method for monitoring and the method for promoting normal development can be used together or independently. By promoting normal development, we prevent onset or progression of disorders associated with premature organs rather than treat disorders associated with the premature organs after they occur.

### DESCRIPTION OF PREFERRED EMBODIMENTS

Definitions

The following terms shall be used to describe the preferred embodiments. In the absence of a specific definition set forth herein, the terms used to describe the present invention shall be given their common meaning as understood by those of ordinary skill in the art.

The term “biomarker of normal development” will be defined as a measurable indicator that proper levels of angiogenesis and/or vasculogenesis are taking place for a fetus or infant for its developmental age, gestational age and/or chronological age.

The term “maternal bodily fluid” will refer to any type of bodily fluid from a pregnant woman, including but not limited to urine, whole blood, serum, plasma, tear film, breast milk.

The term “infant bodily fluid” will refer to any type of bodily fluid from an infant including but not limited to urine, whole blood, serum, plasma, tear film.

The term “developmental age” as used herein, refers to the number of weeks that have passed since the infant's conception. For an “in utero” infant, the “developmental age” will correspond to the infant's gestational age. For a pre-term infant, the “developmental age” will of course encompass both the weeks spent in utero, plus a number of weeks thereafter during which infant development continues “ex utero.”

The “full developmental term” as used herein refers to the normal 38-40 week in utero gestational period for a human infant.

The term “hCG” will refer to any isoform of naturally-occurring or synthetic “human chorionic gonadotropin” including its hyper glycosylated form. The term “hCG” will also refer to any fragment of the hormone that can activate the LH/hCG receptor. The term “LH” will refer to any isoform of naturally occurring or synthetic “luteinizing hormone” or fragment of LH that can activate the LH/hCG receptor. The term LHRH will refer to any isoform of naturally occurring or synthetic “luteinizing hormone releasing hormone” In this patent, when we refer to the use of “active ingredient” or to “hCG/LH receptor activator” we refer to the “hCG”, “LH”, or “LHRH” or a “combination of hCG and/or LH and/or LHRH”.

The term “VEGF 121” refers to vascular endothelial growth factor A, isoform 121. The term VEGF121 will refer to the naturally occurring isoform known as VEGF121 or to any recombinant or synthetic VEGF121.

The term “LH” refers to Luteinizing hormone.

The term “LHRH” refers to Luteinizing hormone releasing hormone.

The term “hCG” refers to human chorionic gonadotropin.

The term “LH/hCG receptor activator” refers to hCG, LH and LHRH individually or combined with one or both of the others.

As used herein, the term “pharmaceutically acceptable carrier,” “diluent,” “additive” or “excipient” means a chemical composition with which the active ingredient either alone or in combination, can be used to administer the appropriate compound(s) to a patient or subject in therapeutic methods according to the preferred embodiments of the or its present invention.

The term “ROP” will stand for “retinopathy of prematurity” (which is also referred to referred to as retrolental fibroplasia” in older medical literature).

The term “patient” or “subject” is used throughout the specification to describe an animal, generally a human, preterm infant. Preterm infants, also known as premature infants, are those who have been born at less than 38 weeks' gestational age (i.e. <38 weeks in utero). Preterm infants born at <30 weeks of gestational age and/or at a birthweight <1500 g and/or other (older) preterm infants who have a complex postnatal medical course, are at highest risk for retinopathy of prematurity. Preterm infants born <34 weeks of gestational age and/or at a birthweight <2000 g are at high risk for several morbidities associated with prematurity.

The term “physiological amount” is used throughout the specification to describe an amount of the LH/hCG receptor activator administered either systemically or to a specific organ at appropriate intervals, to achieve one or more of the following circumstances:
to raise and maintain the patient's LH/hCG combined hormonal levels to the equivalent bioactivity of LH/hCG hormones (adjusted for potency differences between hCG and LH) normally present in fetuses of the equivalent developmental age” (or equivalent gestational age) until the preterm infant reaches the equivalent of full term gestational age.
to raise and maintain the patient's VEGF 121 level to the VEGF 121 level that is physiologically appropriate for patient's “developmental age” until the preterm infant reaches the equivalent of the full term developmental age.
to administer proper dosage of LH/hCG receptor activator to promote overall normal organ development as determined by clinical examination, radiologic, laboratory and/or other investigations.

The term “appropriate intervals” as used herein refers to the intervals of administration of physiological amounts of hCG, and/or LH, and/or LHRH necessary to maintain in the patient a level of angiogenic activity caused by appropriate receptor activation level of the combined LH/hCG receptor activator that is normally present in “in utero” infants of the same “developmental age,” until the preterm infant reaches the equivalent of full term gestational age. The appropriate activation level of the LH/hCG receptor can be determined by the aforementioned criteria.

Discussion

Both LH and hCG activate the same receptor in the human body. Multiple organs of the body contain LH/hCG receptors. A preterm infant is deprived of the placentally-derived hCG exposure that he/she would have had if he/she would have remained in utero, which would have helped the multiple organs of the body develop. Thus, preterm infants experience placental hCG deficiency due to the premature separation of the preterm infant from the placenta. Though many morbidities have been previously linked to premature birth, we are the first to aim to promote the normal development of immature organs using hormonal replacement for placental hCG-deficiency. Though LH and hCG have been previously identified as VEGF regulators in reproductive organs, we are the first to identify hCG and LH as a specific regulator of the VEGF 121 isomer and the first to identify VEGF 121 as the main isoform of VEGFA needed for the overall normal development of the fetus (in utero) as well as the preterm infant (ex utero). Thus, we are the first to identify the potential of VEGF121 as a biomarker for the developmental status of the preterm infant, developmental status of the fetus (in utero) and as well the (c) utility of hCG/LH receptor activator replacement therapy (due to its ability to regulate VEGF 121 production).

In the preferred embodiment, maternal (in the case of a fetus) or preterm infant levels of VEGF121 are monitored, to determine whether appropriate angiogenic activity is occurring to allow preterm infant or fetal development to proceed normally. Preferred methods measure VEGF121 in the bodily fluid of an infant or a pregnant mom (in the case of a fetus) for its use as an appropriate biomarker for this purpose.

The VEGF121 can be measured from the maternal serum or other maternal bodily fluid to monitor angiogenesis and vasculogenesis in the fetus because the VEGF121 levels in the pregnant mother should correspond to fetal levels. Use of VEGF121 as a fetal and pregnancy biomarker has utility in medical diagnostics and prognostics for both the fetus and the pregnancy itself. The use of this biomarker improves risk assessment outcomes beyond that of clinical factors and of uterine/umbilical artery Doppler velocimetry (which is currently in use). If the VEGF121 biomarker is abnormal, the cause of its abnormality could then be investigated and a treatment plan could potentially be put into place.

In the preferred embodiment of the method to promote normal development of immature organs of preterm infants, hCG and/or LH, and/or LHRH (individually or collectively referred to as the LH/hCG receptor activator) are administered to preterm infants in physiological amounts at appropriate intervals to achieve one or more of the following:
to raise and maintain the patient's LH/hCG combined hormonal levels to the equivalent

LH/hCG hormone levels that are normally systemically present in fetuses of the equivalent developmental age until the preterm infant reaches the equivalent of full term developmental (gestational) age.
to raise and maintain the patient's VEGF 121 level to the VEGF 121 level that is physiologically appropriate for patient's developmental age until the preterm infant reaches the equivalent of the full term developmental age.
to administer proper dosage of LH/hCG receptor activator to promote normal organ development as determined by clinical examination, radiologic, laboratory and/or other investigations.

The LH/hCG receptor activator may be administered either locally to a particular fetal organ (such as topically to the eye) or may be administered systemically (such as orally or intravenously). The appropriate dosage and timing of LH/hCG receptor activator treatment will be determined by one or more of the following:
physiologic mean levels of hCG/LH in fetuses of same equivalent developmental age;

the physiologic mean level of hCG/LH approximates the level of hCG and/or LH that the preterm infant would have had if he/she had remained in utero.
patient's VEGF121 level compared to mean VEGF121 levels of other infants of equivalent gestational ages whose development is proceeding normally.
   Serial clinical exam (such as weekly) of preterm infant (i.e. patient) to determine whether dosage is appropriate for normal organ development of individual patient (such as in the case of the eye, weekly dilated retinal examinations by an ophthalmologist to evaluate retinal development or serial (such as weekly) evaluation of the retina by other means such as by optical coherence tomography (OCT).

hCG is a hormone primarily produced by the placenta during pregnancy, though a small additional amount may be produced by kidney of the fetus. (McGregor W G, Kuhn R W, Jaffe R B. Biologically active chorionic gonadotropin: synthesis by the human fetus. Science. 1983;220(4594):306-308). From fetal life through adult life, LH is a hormone produced by the pituitary (both male and female). LH and hCG are highly homologous hormones to one another and activate the same receptor G-coupled LH/hCG receptor. LHRH stimulates the release of LH from the pituitary. Thus, in terms of raising the patient's angiogenic activity to the level of angiogenic activity caused by the activation of the LH/hCG receptor normally present in “in utero” infants of the same “developmental age,” any one of these three active ingredients can be employed. Similarly, any combination of these three active ingredients can be used.

Acknowledging that luteinizing hormone (LH) activates the same receptor in the human body as hCG, and that LHRH causes the endogenous release of LH from the patient, this administration includes the substitution or joint administration of either or both LH or LHRH for hCG. When this is done, LH or LHRH would be administered in an amount equivalent to physiologic levels of hCG (as described above) but adjusted for the difference in potency between hCG and LH.

The organs which often are affected by prematurity include without limitation the eyes, heart, the bronchopulmonary system, brain, colon, kidney, gastrointestinal and immune system. Promoting normal development in the preterm infant will also be effective in preventing the adult onset of hypertension and other lung and cardiac disorders for survivors of preterm birth.

A method for promoting normal development of a specific immature organs of preterm infants involves administering hCG and/or LH, and/or LHRH locally to a target immature organ of a preterm infants in physiological amounts at appropriate intervals to achieve any one or more of the following:
to raise and maintain the patient's LH/hCG combined hormonal levels to the equivalent LH/hCG hormone levels that are normally systemically present in fetuses of the equivalent developmental age of a particular target organ until the preterm infant reaches the equivalent of full term gestational age.
to raise and maintain the patient's VEGF 121 level to the VEGF 121 level that is physiologically appropriate for patient's “developmental age” for a particular target organ until the preterm infant reaches the equivalent of the full term developmental age.
to administer proper dosage of LH/hCG receptor activator to promote normal development of the target organ as determined by clinical examination, radiologic, laboratory and/or other investigations (such as in the case of the eye, weekly dilated retinal exams or weekly retinal evaluated by optical coherence tomography).

As applied specifically to the promotion of normal ocular development, the hCG and/or LH and/or LHRH is/are administered to preterm infants in physiological amounts, and at appropriate intervals, necessary to achieve the one or more of the criteria listed above for administration to a target organ This administration is believed to enhance normal vascularization of the preterm infant's immature retina, and thus prevent the incidence and/or progression of pathologic retinal neovascularization which is associated with ROP.

This approach to preventing ROP is surprising, since hCG and LH are pro-angiogenic agents, known to raise VEGF (vascular endothelial growth factors) levels in several tissue types and late stage ROP is associated with levels of VEGF that are too high.
Babitha V, Yadav V P, Chouhan V S, et al. Luteinizing hormone, insulin like growth factor-1, and epidermal growth factor stimulate vascular endothelial growth factor production in cultured bubaline granulosa cells. Gen Comp Endocrinol. 2014;198:1-12.
Licht P, Russu V, Wildt L. On the role of human chorionic gonadotropin (hCG) in the embryo-endometrial microenvironment: implications for differentiation and implantation. Semin Reprod Med. 2001;19(1):37-47.
Reisinger K, Baal N, McKinnon T, Munstedt K, Zygmunt M. The gonadotropins: tissue-specific angiogenic factors? Mol Cell Endocrinol. 2007;269(1-2):65-80.
Trau H A, Davis J S, Duffy D M. Angiogenesis in the primate ovulatory follicle is stimulated by luteinizing hormone via prostaglandin E2. Biol Reprod. 2015;92(1):15.
   Furthermore, both hCG and LH appear to be associated with worsening of some VEGF-associated ocular disorders. For example, diabetic women are at high risk for worsening of diabetic retinopathy during pregnancy, when maternal hCG levels are high.
Rasmussen K L, Laugesen C S, Ringholm L, Vestgaard M, Damm P, Mathiesen E R. Progression of diabetic retinopathy during pregnancy in women with type 2 diabetes. Diabetologia. 2010;53(6):1076-1083.
   Studies performed in the 1970's show that pituitary ablation (site of LH production) slows the progression of diabetic retinopathy.
Zimmerman B R, Molnar G D. Prolonged follow-up in diabetic retinopathy treated by sectioning the pituitary stalk. Mayo Clin Proc. 1977;52(4):233-237.

Similarly, in my own prior PCT patent application, METHOD FOR PREVENTING OR TREATING OCULAR DISORDERS WO 2016/010786, hCG receptor antagonists are used to prevent activation of the LH/hCG receptor as a method for treating VEGF-associated ocular disorders. hCG significantly increases pathologic retinal neovascularization in a mouse model of oxidative-induced retinopathy (which is a gold standard model for retinopathy of prematurity). The key point here is the appropriate dosing and timing of administration of the active ingredients hCG and LH will determine whether they can prevent VEGF-associated ocular disorders or exacerbate them.

Thus, one may expect these gonadotropins to promote the pathologic retinal neovascularization which is associated with ROP. For example, high levels of VEGF are strongly associated with the progression of several retinal disorders such as ROP, diabetic retinopathy and age related macular degeneration. In fact, anti-VEGF agents (such as bevacizumab) are currently being used to treat the VEGF-associated disorders of diabetic retinopathy and age-related macular degeneration and anti-VEGF agents are also being investigated for treatment of ROP. Yet hCG and LH are the opposite of anti-VEGF agents since these are pro-angiogenic agents. LH/hCG receptor activation has been shown to induce new blood vessel formation with concomitant activation of VEGF expression.

However, in the preferred embodiments of this invention, we are precisely administering to preterm infants the LH/hCG receptor activator in physiological amounts, to simulate the physiologic angiogenesic and vasculogenesic processes that would have occurred had the preterm infant remained in utero and had not been prematurely deprived of placentally-derived hCG. In this way, we promote normal development of the preterm infant's organs including its eye.

### Methods in General

As noted above the invention involves two parts:
a method to monitor the growth and development of preterm infants and fetuses by monitoring whether a normal level of angiogenesis is occurring for the developmental age).
a method to promote normal growth and development of preterm infants.

The preferred method for monitoring the normal growth and development of the preterm infant would be to measure serum VEGF121 levels in the preterm infant, by utilizing a human VEGF121 Elisa kits (human VEGF121 Elisa kits are already commercially available), corrected for total serum protein (such as by Bradford protein assay) on a serial basis (such as daily or weekly). The patient's VEGF121 level can be compared to a range of serum levels of VEGF121 that has been determined to be physiologically appropriate to promote normal angiogenesis/vasculogenesis in infants of the same developmental age.

The physiologically appropriate serum VEGF121 level at any given developmental age of a preterm infant (such as an infant born at 24 weeks after conception, 25 weeks after conception, etc.) will be determined by performing an epidemiologic study as follows: VEGF121 levels (expressed as VEGF121/per total protein content of the bodily fluid) will be measured in a number of preterm infants (males and females) at a number of different developmental ages (The sample size for the number of preterm infants at each development age will be determined by utilizing standard epidemiologic/statistical methodology that incorporates standard deviation). Then statistically significant correlations between VEGF121 levels of a particular bodily fluid (such as serum or urine) with normal development and correlations between VEGF121 levels of a given bodily fluid (such as serum or urine) with abnormal development (such as occurrence of ROP, bronchopulmonary dysplasia, etc.) will be determined. A VEGF 121 level (or range of VEGF121 levels) will become validated as “normal” or “abnormal” for a particular developmental or gestational age.

Alternatively, the serum VEGF121 levels could be expressed per milliliter (instead of per total protein) and then the same type of methodology as described above can be used.

Alternatively, the appropriate physiological serum VEGF121 level could be determined by using cord blood samples from an adequate number of infants born at a variety of gestational ages instead of utilizing blood from infants after birth and undergo the same type of methodology as described above. Other sample types including but not limited to stool, plasma, whole blood, urine, tear film, could be used to monitor VEGF 121 levels in preterm infants and compared to VEGF121 levels in comparable specimen types and then undergo same methodology described above.

The preferred method for monitoring the angiogenic status of the fetus to ensure fetal normal growth and development of the fetus appropriate for the gestational age would be to measure serum VEGF121 levels in maternal blood (pregnant mom's blood), by utilizing a human VEGF121 Elisa kits (human VEGF121 Elisa kits are already commercially available), corrected for total serum protein (such as by Bradford protein assay) on a serial basis throughout pregnancy. Maternal VEGF121 levels should correspond to fetal VEGF121 levels and thus reflect angiogenic activity of the fetus. The maternal VEGF121 serum level can be compared to a range of serum levels of VEGF121 that has been determined to be physiologically appropriate to promote normal angiogenesis/vasculogenesis in fetuses at any gestational age.

During pregnancy, the maternal VEGF121 level should correlate with the corresponding fetal VEGF121 level which should correspond with fetal angiogenesis. The maternal VEGF121 level should also correspond with uterine and placental angiogenesis that occurs during pregnancy. The physiologically appropriate serum VEGF121 level at any given gestational age during pregnancy (such as 24 week old fetus, 25 week old fetus, etc.) will be determined by performing an epidemiologic study as follows: VEGF121 levels (expressed as VEGF121/per total protein content of the maternal blood) will be measured at different times during pregnancies in a number of women who gave birth to normal, healthy infants and had normal, healthy pregnancies as well as a number of women who gave birth to growth restricted infants or to infants with other types adverse outcomes such as cardiac defects (atrial septal defect or ventricular septal defect) or who had pregnancies complicated by conditions such as pre-eclampsia.

The sample size for the number of pregnant women to take part in the study will be determined by utilizing standard epidemiologic/statistical methodology that incorporates standard deviation. Then statistically significant correlations between VEGF121 levels of a particular maternal bodily fluid (such as serum or urine) with normal pregnancy and normal infant outcomes and correlations between VEGF levels of a given bodily fluid (such as maternal serum or urine) with complicated pregnancies (such as pre-eclampsia) and/or infants with adverse outcomes will be determined such as infants with cardiac defects. A VEGF 121 level (or range of vEGF121 levels) will become validated as “normal” or “abnormal” for fetal growth and pregnancy health.

The preferred embodiment to promote normal growth and development of preterm infants involves the administration of physiological amounts of LH/hCG receptor activators to preterm infants, at appropriate intervals, necessary to raise the patient's angiogenic activity to the to the level that is normally present systematically (or in specific organs) in “in utero” infants of the same “developmental age,” for the remaining developmental term of the pre-term infant. This can be accomplished in one or more of the following ways:
to raise and maintain the patient's LH/hCG combined hormonal levels to the equivalent LH/hCG hormone levels (e.g. serum levels) that are normally systemically present in fetuses of the equivalent developmental age, or that are normally present in a particular target organ until the preterm infant reaches the equivalent of full term developmental (gestational) age
to raise and maintain the patient's VEGF 121 level to the VEGF 121 level that is physiologically appropriate for patient's developmental age either systemically (such as serum levels) or to a level appropriate for a particular target organ until the preterm infant reaches the equivalent of the full term developmental age.
to administer proper dosage of LH/hCG receptor activator to promote normal development of the target organ as determined by clinical examination, radiologic, laboratory and/or other investigations of the patient

The preferred method for estimating the physiological amount of each ingredient or combination of ingredients will be to first estimate the mean serum level of hCG, LH and/or LHRH at various gestational ages from an adequate sample size of human infants to determine appropriate dosing (for systemic dosing). To accomplish this, we could measure the fetal cord blood levels of hCG, LH and LHRH (such as by performing measurements by ELISA testing) from a number of infants who had been born at a variety of gestational ages (i.e. At gestational ages: 24 wks., 25 wks., 26 wks . . . 38 weeks, 39 weeks, 40 weeks.) and normalize cord blood levels for total protein (as can be performed by Bradford protein assay). This will give an estimate of the physiological amount of these hormones that is appropriate for blood for each week of gestation and allow us to estimate appropriate dose of hCG/LH hormone receptor. The cord blood is the most appropriate sample type to estimate fetal levels from within the womb. The appropriate sample size needed for each gestational age would be determined by standard statistical methodology.

Alternatively, or in addition, the LH/hCG receptor activators could be dosed in physiologic doses and appropriate intervals so that physiologic VEGF121 levels for the correct gestational age is achieved (Method to determine appropriate VEGF121 level is described above).

Alternatively, and/or in addition, clinical exam, radiologic studies and other lab investigations could be utilized to determine or to fine tune the correct dosage and timing of the LH/hCG receptor activators. Clinical exam could be performed on a daily or weekly basis and may be enhanced periodically by x-rays, mri/ct scan, ultrasound evaluations, ocular coherent tomography or laboratory measures (such as C-reactive protein) as deemed appropriate by an experienced neonatologist or other pediatric specialist.

In the case of direct ocular administration (such as eye drops) to promote eye development, we would determine the amount of LH (or hCG animal equivalent) and VEGF 121 (animal equivalent) that is normally present in neonatal animal models who are naturally born with immature eyes such as rabbits and mice and determine how the levels of the LH/hCG receptor activator changes as the neonatal eye matures. If human fetal eyes are available (from legally aborted fetuses whose parents have consented to have the fetal eyes donated to research and a medical ethics review board approves a study), then the LH/hCG levels and VEGF121 levels of the human fetal eyes themselves can be measured. Then based on drug permeability studies and pharmacokinetic studies, the dosing and timing of physiologic levels of LH/hCG receptor activators for human preterm infants can be determined. The dosing and timing of the dosing can be fine-tuned based upon clinical exam (serial retinal exam) of the patient. In other words, clinical study (with weekly dilated retinal exams) would determine what amount of dose and timing of dosing that promotes optimal eye development of the retinal vasculature.

However, these methods assume that the birth level of hCG, LH and LHRH will remain at that level if not supplemented with physiological doses at various developmental ages thereafter. This assumption ignores the possibility that some preterm neonates self-compensate for their hCG deficiency with upregulation of their pituitary production of the homologous hormone, LH. Numerous other factors may cause variations from standard, such as body weight, other health factors, etc. Human clinical trials of preterm infants will be performed to determine the safest and most appropriate dosing of these active ingredients to simulate physiologic combined levels of the hormones or to achieve serum, tear film or urinary levels of VEGF121 (expressed per milliliter or per mg of total protein).

Another possibility is that therapeutic dosing will need to be individualized based upon each infant's own serum levels of these hormones. If an infant is found to be deficient in a particular hormone (such as hCG or LH), the active ingredient will be administered in a dosage to supplement the infant's own natural hormone level. The determination of supplemental dosing may be performed weekly or at a different time intervals to be determined after clinical testing. The sampling might be done weekly, for example, but the levels determined might indicate no variation from the standard at that interval, making administration of additional hCG, and/or LH and/or LHRH unnecessary at that time.

A calculation of the physiological dosage of hCG or LH will consider the fact that both hormones activate the same receptor. If LHRH is to be administered, the dosage of it will take into account that LHRH raises the LH level through the increased pituitary release of LH. The physiological amount of active ingredient may need to be determined on a weekly basis as the infant matures and the eyes approaches full vascularization and other organs of the body continue to develop ex utero until they reach the point of maturation they would have reached if the infant had remained in utero until full maturation (approx. 40 weeks of gestational age). The dosage may be based on administering the correct combination of hormones to achieve mean VEGF121 levels (either expressed per milliliter or per total protein) appropriate for the developmental age of the preterm infant. For promoting ocular development, routes of administration for the treatment may include but are not limited to: topical (such as in the form of an eye drop or ointment), intraocular, subconjunctival, intravitreal, any form of intraocular administration, oral (such as added to formula) or intravenous. Topical application in the form of eye drops is preferred when aim is normal ocular development to prevent ROP alone.

For administration by eye drops or ointment, one must consider the percentage absorption of the LH/hCG receptor activator into the posterior chamber of the eye and its rate of release from the vitreous gel (in which the drug may be stored). The medication may potentially be incorporated with an ocular transporter to enhance the rate of absorption of the active ingredients into the posterior chamber of the eye.

For promoting normal development of the immature organs system wide, systemic administration is used, including without limitation, intrathecal, intravenous, parenteral, intraperitoneal, oral, intranasal administration. Oral or intravenous route is preferred when treatment is to be directed to prevent hCG-deficiency syndrome for multiple morbidities and promote normal development of multiple organs.

For oral administration, the effects of digestion and absorption must be considered as well as the medication's ability to penetrate the blood-retinal barrier. Bioavailability of the actives will vary with the route of administration, and must be considered.

In the case of LHRH, intranasal administration might be preferred so that LHRH can more easily reach the brain to increase LH production by the pituitary. Route of administration will be an important determinant in physiological amount of dosing.

The formulations of the pharmaceutical compositions described herein may be prepared by any method known or hereafter developed in the art of pharmacology. The actives will be combined with suitable “pharmaceutically acceptable carriers,” “diluents,” “additive” or “excipients.” One or more additional bioactive agents may be incorporated into the pharmaceutical formulation. In general, such preparatory methods include the step of bringing the active ingredient into association with a carrier or one or more other accessory ingredients, and then, if necessary or desirable, shaping or packaging the product into a desired single or multi-dose unit.

1. A method for monitoring fetal and/or preterm infant development and treating as necessary, comprising: first monitoring bodily fluid levels of VEGF121 to determine initially monitored VEGF121 levels; comparing said initially VEGF121 levels to those normally present in fetuses or infants of the equivalent developmental age to determine whether said initially monitored VEGF121 levels indicate that appropriate angiogenic activity is occurring to allow development to proceed normally; and administering a therapeutic to said pregnant woman or preterm infant in physiological amounts at appropriate intervals, to adjust its VEGF 121 levels to those normally present in fetuses/infants of the equivalent developmental age until said infant reaches the equivalent of its full term developmental age.

2. The method of claim 1 in which said initially monitored level of VEGF121 is measured in the bodily fluids of a preterm infant.

3. The method of claim 2 in which said level of VEGF 121 is corrected for total amount of protein in bodily fluid so that the VEGF121 level is expressed as VEGF121/total protein in bodily fluid.

4. The method of claim 2 in the level of VEGF is corrected for the total amount of bodily fluid in the preterm infant so that the VEGF121 level is expressed as VEGF121/milliliter of bodily fluid.

5. The method of claim 1 in which the levels of VEGF121 are measured in the bodily fluids of the pregnant mother.

6. The method of claim 5 in which the level of VEGF 121 is corrected for total amount of protein in bodily fluid so that the VEGF121 level is expressed as VEGF121/total protein in bodily fluid.

7. The method of claim 5 in which the level of VEGF 121 is expressed as VEGF121/milliliter of bodily fluid.

8. A method for monitoring angiogenesis and vasculogenesis of the pregnant woman and treating as necessary, comprising: monitoring bodily fluid levels of VEGF121 of a pregnant woman, to determine whether normal angiogenesis and vasculogenesis during pregnancy is occurring; and administering a therapeutic to said pregnant woman or preterm infant in physiological amounts at appropriate intervals, to adjust its VEGF 121 levels to those normally present in fetuses/infants of the equivalent developmental age until said infant reaches the equivalent of its full term developmental age.

9. The method of claim 8 in which the level of VEGF 121 is corrected for total amount of protein in bodily fluid so that the VEGF121 level is expressed as VEGF121/total protein in bodily fluid.

10. The method of claim 8 in which the level of VEGF is corrected for the total amount of bodily fluid in the preterm infant so that the VEGF121 level is expressed as VEGF121/milliliter of bodily fluid.

11-26. (canceled)
